(19) United States
(12) Patent Application Publication
(10) Pub. No.: US 2009/0105510 A1
(43) Pub. Date: Apr. 23, 2009

(54) Fluorination catalysts, method for their preparation, and method for producing fluorinated compounds using the catalysts

(75) Inventors: Heng-dao Quan, Tsukuba-shi (JP); Huie Yang, Tsukuba-shi (JP); Masanori Tamura, Tsukuba-shi (JP); Akira Sekiya, Tsukuba-shi (JP)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: NATIONAL INSTITUTE OF ADVANCED INDUSTRIAL SCIENCE AND TECHNOLOGY, Tokyo (JP)

(21) Appl. No.: 12/314,803

(22) Filed: Dec. 17, 2008

Related U.S. Application Data
(62) Division of application No. 11/456,126, filed on Jul. 7, 2006.

(30) Foreign Application Priority Data
Jul. 7, 2005 (JP) 2005-199350

(51) Int. Cl. C07C 17/20

(52) U.S. Cl. 570/160; 570/161

(57) ABSTRACT

A method for the preparation of a novel fluorinated compound, particularly 1,1,1,3,3-pentafluoropropane (HFC-245fa), by using a fluorination catalyst obtained by treating a metal salt containing a chromium salt such as chromium oxide with chlorine gas and/or oxygen gas. Examples of the metal salt may include, besides a chromium salt, one or more catalytically active metal salts selected from magnesium salts, aluminum salts, zinc salts, sodium salts, nickel salts, iron salts, cobalt salts, vanadium salts, manganese salts and copper salts.

This application is a divisional of application Ser. No. 11/456,126, filed on Jul. 7, 2006 which claims the priority of Japanese Application No. 2005-199350, filed Jul. 7, 2005, which are incorporated herein by reference.

### BACKGROUND OF THE INVENTION

1. Field of the Invention

The present invention relates to a fluorination catalyst mainly containing chromium for producing fluorinated alkanes such as 1,1,1,3,3-pentafluoropropane (HFC-245fa), a method for the preparation of the fluorination catalyst, and a method for producing various fluorinated alkanes using said fluorination catalyst.

2. Description of the Related Art

Freon gas (chlorofluorocarbon) is a highly stable material, is inflammable, non-explosive and harmless to humans. It is therefore frequently used in cooling mediums such as car air conditioners, room air conditioners, refrigerators, foaming agents for urethane resins, foaming agents for hair cosmetic mousses and detergents for IC chips.

This Freon gas, however, has posed the problem that it destroys the ozone layer in the stratosphere resulting in the ozone hole in recent years. In view of this situation, fluorine compounds, excluding chlorine, have become widespread as a substitute for Freon.

A typical substitute for Freon is 1,1,1,3,3-pentafluoropropane (HFC-245fa).

This 1,1,1,3,3-pentafluoropropane is a compound that never destroys the ozone layer, has an allowable level of global warming parameter (GWP) (see, for example, IPCC 3^{rd} Report (2001), Climate Change 2001 (the Scientific Basis), pp 388) and does not belong to the volatile organic compounds (VOC) (see, for example, M. C. Bogdam, D. J. Williams, and P. Verbiest. “Utilization of HFC-245fa in Spray Form” presented at Polyurethanes Expo '98, Sep. 17-20, 1998).

Moreover, HFC-245fa is a non-flammable material that has low toxicity and a boiling point of 15.3° C. (see, for example, Sekiya, Akira, Misaki, Susumu, J. Fluorine Chem. 101 (2000) 215-222 and Brisdon, Alan K.; Crossley, Ian R., Chem. Commun., 20, 2002, 2420-2421).

Though HCFC-141b is a useful foaming agent in heat insulating materials, its use will be totally abolished by 2020 in advanced nations. Since HFC-245fa has the aforementioned characteristics, it is expected to be a substitute for HCFC-141b (see, for example, Peter B. Logsdon, David J. Williams, The next Step: Commercialization timeline for HFC-245fa, Honeywell report).

With regard to methods of producing HFC-245fa, various methods have been reported (see, for example, Izvest. Akad. Nauk S. S. S. R. Otdel. Khim. Nauk. 1960, 142. and N. Tatsuo, Y. Akinori, S. Noriaki, S. Takashi, WO40335 (1998) and U.S. Pat. No. 5,563,304) and a large number of various technologies have been reported also in the publications of patents.

Publication of WO01/036355 discloses a method for the preparation of 1,1,1,3,3-pentafluoropropane, the method comprising a liquid phase reaction process in which 1,1,1,3,3-pentahalopropane (provided that at least one halogen is not F) is fluorinated by HF in the presence of an antimony pentahalide catalyst in a reactor to obtain a reaction product including at least 1,1,1,3,3-pentafluoropropane and antimony pentahalide catalyst, wherein the fluorination is carried out at a temperature less than 50° C.

Publication of WO01/056961 discloses a method for the preparation of a hydrogen-containing fluorinated hydrocarbon, the method comprising a process in which a hydrogen fluoride reaction raw material is used to run a liquid phase fluorination reaction with one or more halogenating hydrocarbon reaction raw materials (for example, 1,1,1,3,3-pentachloropropane, 1,3,3,3-tetrachloropropene and 1-chloro-3,3,3-trifluoropropene) selected from the group consisting of chlorinated alkenes and hydrogen-containing chlorinated alkanes in the presence of a fluorination catalyst in a reactor to obtain a reaction mixture containing 1,1,1,3,3-pentafluoropropane as the reaction products, wherein, as the reactor, a reactor is used in which at least a part which can be brought into contact with the reaction mixture is made of an alloy material constituted of 18 to 20% by weight of chromium, 18 to 20% by weight of molybdenum and 1.5 to 2.2% by weight of at least one element selected from niobium and tantalum which is balanced with nickel.

Publication of Japanese Patent Application Publication (Laid-Open) No. 8-511271 discloses a method for the preparation of 1,1,1,3,3-pentafluoropropane (CF₃CH₂CF₂H), the method comprising reacting CF₃CH₂CF₂Cl with hydrogen in the presence of a reducing catalyst selected from nickel, palladium, platinum and rhodium.

Publication of JP-A No. 7-69943 discloses a method for the preparation of 1,1,1,3,3-pentafluoropropane, the method comprising reducing 1,1,3,3,3-pentafluoropropene by reacting it with hydrogen in the presence of a palladium catalyst using a vapor phase method at a temperature range from 40 to 300° C.

Publication of JP-A No. 7-138194 discloses a method for the preparation of 1,1,1,3,3-pentafluoropropane, the method comprising reducing 3-chloro 1,1,1,3,3-pentafluoropropane by hydrogen in the presence of a novel metal catalyst selected from the group consisting of palladium, platinum and rhodium in a vapor phase.

Publication of JP-A No. 8-73385 discloses a method for the preparation of 1,1,1,3,3-pentafluoropropane, the method comprising a process in which 1,1-difluoroethylene is reacted with dichlorofluoromethane in the presence of a Lewis acid catalyst constituted of a halide or halogenated oxide of at least one element selected from B, Al, Ga, In, Fe, Ni, Co, Sb, Nb, Sn, Ti, Zr, Hf, W and Ta to obtain 1,2,2-trihydrodichlorotrifluoropropane, which is then fluorinated by hydrogen fluoride.

Publication of JP-A No. 2001-261593 discloses a method for the preparation of a hydrogen-containing fluorinated hydrocarbon to obtain a reaction mixture containing a hydrogen-containing fluorinated hydrocarbon as a reaction product by reacting a hydrogen fluoride reaction raw material with one or more halogenated hydrocarbon reaction raw materials selected from the group consisting of a chlorinated alkene and a hydrogen-containing chlorinated alkane in the presence of one or more fluorination catalysts selected from the group consisting of SbF₅, SbCl₅, SbCl₂F₃, NbClF₄, NbF₅, TaCl₅, TaF₅ and TaClF₄, the method comprising a step of supplying one of these reaction raw materials to the space between an inside reactor constituted of a material substantially standing against the reaction and an outside reactor which is disposed outside of the inside reactor and substantially has a resistance to at least one of the reaction raw materials; a step of supplying the one reaction raw material to the inside reactor; a step of supplying the other reaction raw material to the inside reactor; and a reaction step of reacting both the reaction raw materials under the presence of a fluorination catalyst in the inside reactor to obtain a reaction product containing a hydrogen-containing fluorinated hydrocarbon.

Publication of JP-A No. 2000-63302 discloses a method for the preparation of a fluorinated propane represented by the formula (2):

C₃HⱼFₖX₁ (2)

wherein Xs respectively represent a chlorine atom, a bromine atom or an iodine atom, j denotes an integer from 1 to 6, k denotes an integer from 2 to 7, l denotes an integer from 0 to 5, provided that j+k+l=8; by fluorinating a halogenated propane represented by the formula (1):

C₃HₐF_{b}X_{c} (1)

wherein Xs respectively represent a chlorine atom, a bromine atom or an iodine atom, a denotes an integer from 1 to 6, b denotes an integer from 0 to 6, c denotes an integer from 1 to 7, provided that a+b+c=8, in a vapor phase under the presence of a fluorination catalyst, wherein the catalyst is one produced by supporting an antimony compound on activated carbon.

Publication of JP-A No. 8-104655 discloses a method for the preparation of 1,1,1-3,3-pentafluoropropane is reported in which 1,1,1,3,3-pentachloropropane is reacted with hydrofluoric acid anhydride under the presence of a catalyst in a liquid phase.

Here, the catalyst is oxides, halogenated oxides or halides of derivatives of metals of the main groups IIIa, IVa and Va and the subgroups IVb, Vb and VIb and more specifically chlorides, fluorides, chlorofluorides of antimony or mixtures of these compounds.

Publication of Japanese Patent Application No. 7-65159 discloses a method for the preparation of 1,1,1,3,3-pentafluoropropane, the method comprising a step A of reducing 2,3-dichloro-1,1,1,3,3-pentafluoropropane by a vapor phase reaction using hydrogen under a pressure higher than the atmospheric pressure in the presence of a hydrogenating catalyst made of palladium, a step B of introducing all products obtained in the above step A into a condenser to obtain a component consisting of hydrogen and hydrogen chloride as a non-condensed component and a component consisting of 1,1,1,3,3-pentafluoropropane as a condensed component, or a component consisting of hydrogen as a non-condensed component and a component consisting of hydrogen chloride and 1,1,1,3,3-pentafluoropropane as a condensed component, a step C of separating hydrogen from the non-condensed component obtained in the above step B to recycle the separated hydrogen to the above step A and a step D of separating the 1,1,1,3,3-pentafluoropropane from the condensed component obtained in the step B.

Publication of Japanese Patent Application No. 7-44094 discloses a method for the preparation of 1,1,1,3,3-pentafluoropropane, the method comprising fluorinating 1,1,1,3,3-pentachloropropane in a liquid phase by using hydrogen fluoride under the presence of antimony pentahalide catalyst.

Publication of JP-A No. 8-337542 discloses a method for the preparation of 1,1,1,3,3-pentafluoropropane, the method comprising reducing one or more hydrocarbonchlorofluorides selected from CF₃CClₓCClF₂ (wherein X represents a H atom or a Cl atom) and CF₃CCl═CF₂ by using hydrogen in the presence of a reducing catalyst using, as a first component, one or more metals selected from Ru, Rh, Pd and Pt and as a second component, one or more metals selected from Ni, Co, La, Re, W, Ta, Nb, Ti, Zr, Mo, Cu, Ag and Au.

Publication of JP-A No. 9-2983 discloses a method for the preparation of 1,1,1,3,3-pentafluoropropane, the method comprising fluorinating 1,1,1,3,3-pentachloropropane in a liquid phase by using hydrogen fluoride under the presence of a fluorination catalyst constituted of at least one element selected from Sb, Nb, Ta and Sn.

Publication of JP-A No. 9-67281 discloses a method for the preparation of 1,1,1,3,3-pentafluoropropene, the method comprising bringing 1,1,1,3,3,3-hexafluoropropane into contact with an oxide of trivalent chromium and/or an oxide of partially fluorinated trivalent chromium under the presence of oxygen in a gas state to dissociate hydrofluoric acid.

Publication of JP-A No. 9-110737 discloses a method for the preparation of 1,1,1,3,3-pentafluoropropane, the method comprising reacting a halogenated propane represented by the formula CX₃CH₂CHX₂ (wherein X represents a fluorine atom or a chlorine atom, provided that Xs are not fluorine atoms at the same time) with hydrofluoric acid anhydride in the presence of an antimony catalyst constituted of pentavalent or trivalent antimony to obtain 1,1,1,3,3-pentafluoropropane.

Publication of JP-A No. 9-241188 discloses a method for the preparation of 1,1,1,3,3-pentafluoropropane, the method comprising fluorinating 1-chloro-3,3,3-trifluoropropene by hydrogen fluoride in a liquid phase under the presence of antimony pentahalide catalyst.

Publication of JP-A No. 9-268139 discloses a method for the preparation of 1,1,1,3,3-pentafluoropropane, the method comprising reacting 1,1,1,3,3-pentachloropropane by hydrogen fluoride in a vapor phase under the presence of a fluorination catalyst constituted of a chromium-carrying alumina catalyst.

Publication of JP-A No. 9-268140 discloses a method for the preparation of 1,1,1,3,3-pentafluoropropane, the method comprising reacting 1-chloro-3,3,3-trifluoropropene with hydrogen fluoride in a vapor phase under the presence of chromium-carrying alumina catalyst.

Publication of JP-A No. 9-268141 discloses a method for the preparation of 1,1,1,3,3-pentafluoropropane, the method comprising supplying 1,1,1,3,3-pentachloropropane and/or hydrogen fluoride continuously to a reaction area when fluorinating 1,1,1,3,3-pentachloropropane by hydrogen fluoride in a liquid phase under the presence of antimony pentahalide catalyst to produce 1,1,1,3,3-pentafluoropropane.

Publication of JP-A No. 10-17502 discloses a method for the preparation of 1,1,1,3,3-pentafluoropropane is reported, the method comprising adding hydrogen fluoride to 1,3,3,3-tetrafluoropropene in the presence of a hydrogen halide-adding catalyst (halide of one or more metals selected from aluminum, tin, bismuth, antimony and iron).

Publication of JP-A No. 10-72381 discloses a method for the preparation of 1,1,1,3,3-pentafluoropropane is reported, the method comprising adding hydrogen fluoride to 1-chloro-3,3,3-trifluoropropene in the presence of an addition catalyst (antimony pentahalide or boron trihalide) to obtain 1,1,1,3-tetrafluoro-3-chloropropane, which is then disproportionated in the presence of a disproportionating catalyst (antimony pentahalide or aluminum halide).

Publication of JP-A No. 10-72382 discloses a method for the preparation of 1,1,1,3,3-pentafluoropropane is reported, in which 1,1,1-3,3-pentachloropropane is fluorinated in a liquid phase by hydrogen fluoride under the presence of a fluorination catalyst containing a halide of one or more elements selected from antimony, niobium and tantalum, the method comprising supplying 1,1,1,3,3-pentachloropropane continuously together with hydrogen fluoride to a reactor and with drawing the fluorinated products continuously from the vapor phase, wherein 1,1,1-3,3-pentachloropropane is reacted in a concentration of 200 mol % or less based on the fluorination catalyst existing in the reaction system.

Publication of JP-A No. 10-87523 discloses a method for the preparation of 1,1,1,3,3-pentafluoropropane is reported, the method comprising a first stage in which 1-chloro-3,3,3-trifluoropropene (CF₃CH═CHCl) is substantially produced and a second stage in which 1-chloro-3,3,3-trifluoropropene is converted into 1,1,1-3,3-pentafluoropropane (CF₃CH₂CHF₂) in a liquid phase.

Publication of JP-A No. 10-101594 discloses a method for the preparation of 1,1,1,3,3-pentafluoropropane, the method comprising fluorinating 1,1,1,3,3-pentachloropropane by hydrogen fluoride in the presence of a fluorination catalyst containing, as a first component, a halide of pentavalent antimony and as a second component, another metal halide.

Publication of JP-A No. 11-158089 discloses a method for the preparation of 1,1,1,3,3-pentafluoropropane is reported, in which a compound A selected from 1,1,1,3,3-pentachloropropane (F240fa), a derivative obtained by fluorinating a part of F240fa, 1,1,3,3-tetrachloro-1-propene (F1230za) and 1,3,3,3-tetrachloro-1-propene (F1230zd) with hydrogen fluoride in a liquid phase, wherein a compound containing titanium as its base is used as a catalyst.

Publication of JP-A No. 11-180908 discloses a method for the preparation of 1,1,1,3,3-pentafluoropropane, in which 1,1,1,3,3-pentachloropropane is reacted with hydrogen fluoride in the presence of no catalyst to obtain an intermediate constituted mainly of 1,1,1-trifluoro-3-chloropropene and/or 1,1,1-tetrafluoropropene and this intermediate is fluorinated in the presence of a catalyst (fluoride of chromium or its oxyfluoride) to obtain 1,1,1,3,3-pentafluoropropane.

Publication of JP-A No. 2000-95714 discloses a method for the preparation of 1,1,1,3,3-pentafluoropropane and/or 1-chloro-3,3,3-trifluoropropene, in which when one or more raw materials selected from 1,1,1,3,3-pentachloropropane, 1,1,3,3-tetrachloropropene and 1,3,3,3-tetrachloropropene is fluorinated by hydrogen fluoride in the presence of a catalyst (chromium oxyfluoride catalyst and/or aluminum oxy fluoride catalyst), dehydrated ones are used as the above raw materials and hydrogen fluoride to be supplied to the reaction system.

Publication of JP-A No. 2000-143561 discloses a method for the preparation of 1,1,1,3,3-pentafluoropropane is reported, in which 1-chloro-3,3,3-trifluoropropene is fluorinated in a vapor phase by hydrogen fluoride in a reaction area where a catalyst constituted of activated carbon carrying a metal having a high-atomic valence selected from halides of antimony, tantalum, niobium, molybdenum, tin or titanium exists.

Publication of JP-A No. 2001-58967 discloses a method for treating 1,1,1,3,3-pentafluoropropane, in which 1-chloro-3,3,3-trifluoropropene and/or 1,3,3-pentafluoropropane containing unsaturated impurities constituted of other halogenated hydrocarbons having an unsaturated bond is brought into contact with chlorine gas in the presence of an activated carbon catalyst to convert the above unsaturated impurities into chlorine adducts, thereby dropping the content of these impurities.

Publication of JP-A No. 2003-176243 discloses a method for the preparation of, 1,1,3,3-pentafluoropropane and/or 1,1,3,3,3-pentafluoropropene, the method comprising reducing 2-chloro-1,1,3,3,3-pentafluoropropene by reacting it with hydrogen in a vapor phase in the presence of a catalyst constituted of at least one type selected from palladium, platinum and rhodium (catalyst constituted of at least one type selected from palladium, platinum and rhodium carried on a support constituted of at least one type selected from activated carbon, alumina, silica gel, titanium oxide and zirconia).

Japanese Patent Application No. 8-504369 discloses a method for the preparation of 1,1,1,3,3-pentafluoropropane is reported, the method comprising a process of reacting a compound represented by the formula CF_{y}Cl_{3-y}CH₂CHF_{w}Cl_{2-w} (wherein w denotes 0 or 1 and y denotes 0 to 3) with hydrogen fluoride in the presence of a fluorination catalyst selected from the group consisting of a pentavalent antimony halide, pentavalent niobium halide, pentavalent arsenic halide and pentavalent tantalum halide, pentavalent antimony halide mixture, pentavalent niobium halide mixture, pentavalent arsenic halide mixture and pentavalent tantalum halide mixture and mixtures of these halides under the condition satisfactory to produce a compound represented by the formula CF₃CH₂CF₂H.

The methods of producing 1,1,3,3,3-pentafluoropropene are largely classified into a liquid phase catalytic fluorination method and a vapor phase catalytic fluorination method.

The liquid phase catalytic fluorination method is carried out by fluorinating 1,1,1,3,3-pentachloropropane (PCP) utilizing anhydrous hydrogen fluoride (AHF). In this liquid phase catalytic fluorination, SbCl₅ is used as a catalyst and HFC-245fa is obtained at a high yield. However, this process causes strong corrosion of a reactor, posing the problem that tar-like products deteriorate the activity of the catalyst (see, for example, N. Keiichi, O. Shuichi, JP10101593 (1998) and U.S. Pat. No. 5,574,192).

It is reported that in the production method using the vapor phase catalytic fluorination, on the other hand, SbCl₅ carried on carbon has high activity and selectivity as a catalyst (see, for example, Publication of JP No. 3031465).

However, such a catalyst carried on carbon has a problem concerning stability at high temperatures and also, the problem that it is difficult to regenerate the catalyst when carbonization takes place.

On the contrary, the inventors of the present invention have made it clear that SbCl₅ (or antimonate) carried on an inert porous metal fluoride can be used as a catalyst to produce HFC-245fa and filed a patent application based on this finding (Patent Application No. 2004-064929). However, this is still unsatisfactory from the viewpoint of catalyst activity and selectivity in the production of HFC-245fa.

In the meantime, chromium salts are known to be fluorination catalysts exhibiting high thermal stability and high activity. However, this is still unsatisfactory from the viewpoint of catalytic activity and selectivity in the production of HFC-245fa (see, for example, U.S. Pat. No. 6,316,681 B1 and U.S. Pat. No. 6,018,084).

As mentioned above, the conventional vapor phase catalytic fluorination method is used industrially as a method for synthesizing fluorine-containing compounds. However, When a chromium salt or a fluorinated antimony-carrying catalyst is used, this method has the drawbacks that in the case of the chromium salt catalyst, insufficient activity is obtained; and, in the case of the fluorinated antimony-carrying catalyst, there is unsatisfactory stability and low activity depending on the carrying support.

### SUMMARY OF THE INVENTION

Therefore, it is an object of the present invention to provide a novel fluorination catalyst that has high stability at high temperatures, is easily regenerated and is superior in catalytic activity and selectivity and to provide a method for the preparation of the fluorination catalyst. Another object of the present invention is to provide a method for the preparation of a novel fluorinated alkane, and particularly, 1,1,1,3,3-pentafluoropropane (HFC-245fa), by using the catalyst.

The inventors of the present invention have repeatedly made studies to solve the above problem and, as a result, found that when a metal salt containing a chromium salt as its major component is treated using chlorine or oxygen at high temperatures, the resulting material has high activity as a fluorination catalyst, and exhibits high catalytic activity and selectivity as a catalyst in the production of HFC-245fa. Thus, the present invention was completed.

These objects of the present invention can be attained by the following embodiments.

The first aspect of the present invention is a fluorination catalyst obtained by treating a metal salt containing a chromium salt or chromium oxide with chlorine gas and/or oxygen gas.

The catalyst can be obtained by treating a metal salt containing a chromium salt or chromium oxide with chlorine gas.

The chromium salt can be a halogenated chromium oxide, chromium nitrate, chromium sulfate, halogenated chromium, chromium perchlorate, chromium acetate, chromium acetylacetonate or a mixture of these compounds.

The chromium salt also can be a halogenated chromium oxide represented by CrCl₃ or CrOₐX_{b} (wherein X represents a halogen atom, a denotes 0 or 1 to 3 and b denotes 0 or 1 to 6, provided that 3≦a+b≦6) or a mixture of these compounds.

The metal salt described above can be one or more other catalytically active metal salts selected from a magnesium salt, an aluminum salt, a zinc salt, a sodium salt, a nickel salt, an iron salt, a cobalt salt, vanadium salt, manganese salt and a copper salt or a metal oxide.

The above other catalytically active metal salt can be a halide, a nitrate, a sulfate, a silicate, a perchlorate, an acetate or an organic acid salt of magnesium, aluminum, zinc, sodium, nickel, iron, cobalt, vanadium, manganese or copper.

The above other catalytically active metal salt can be one or more metal salts selected from halides of magnesium, aluminum and zinc.

The above other catalytically active metal salt can be a zinc halide and the content of zinc is 0.1% by weight to 10% by weight based on all metal salts.

The above other catalytically active metal salt can be an aluminum halide and the content of aluminum is 0.1% by weight to 20% by weight based on all metal salts.

The above other catalytically active metal salt can be a magnesium halide and the content of magnesium is 0.1% by weight to 10% by weight based on all metal salts.

The temperature of the treatment using chlorine gas and/or oxygen gas can be 100° C. to 500° C.

The metal salt described in the above 1 can be a metal salt prepared by a coprecipitation method or an impregnation method.

The second aspect of the present invention is a method for the preparation of a fluorinated alkane or a fluorinated alkene, the method comprising reacting an alkane or an alkene having at least one halogen atom, selected from the group of chlorine, bromine and iodine, and 1 to 4 carbon atoms with hydrogen fluoride in the presence of the fluorination catalyst.

The alkane or alkene having at least one halogen atom and 1 to 4 carbon atoms is a propane or propene halide having 3 carbon atoms and represented by the formula CF_{y}Cl_{3-y}CH₂CHF_{w}Cl_{2-w} (wherein w denotes 0 or 1 and y denotes 0 to 3).

The above fluorinated alkene can be 1-chloro-3,3,3-trifluoropropene and/or 1,3,3,3-tetrafluoropropene produced by reacting 1,1,1,3,3-pentachloropropane with hydrogen fluoride in the presence of the fluorination catalyst.

The above fluorinated alkane can be 1,1,1,3,3-pentafluoropropane produced by reacting 1-chloro-3,3,3-trifluoropropene or 1,3,3,3-tetrafluoropropene with hydrogen fluoride in the presence of the fluorination catalyst.

Another aspect of the present invention is a method for the preparation of a fluorination catalyst, the method comprising preparing one or more chromium salts selected from the group of a halogenated chromium oxide, chromium nitrate, chromium sulfate, halogenated chromium, chromium perchlorate, chromium acetate and chromium acetylacetonate, other catalytically active metal salts selected from the group of the chromium salts and halides, nitrates, sulfates, silicates, perchlorates, acetates and organic acid salts of magnesium, aluminum, zinc, sodium, nickel, iron, cobalt, vanadium, manganese and copper by a coprecipitation method or an impregnation method, and treating these salts at 100° C. to 500° C. in chlorine gas and/or oxygen gas.

### EFFECT OF THE INVENTION

The fluorination catalyst of the present invention is stable at high temperatures and enables the preparation of fluorinated compounds such as intermediate raw materials used to synthesize HFC-245fa or HFC-245fa at a high conversion rate and selectivity.

The porous metal fluoride of the present invention enables the easy production of a highly stable and highly active novel catalyst by chlorinating a chromium salt such as porous fluorinated chromium. The result is a fluorination catalyst that is most important in fluorine chemical industries for synthesizing a fluorine-containing compound. Therefore, if the fluorination catalyst of the present invention is used, cooling mediums, detergents and foaming agents that are made of materials substituted for Freon can be produced efficiently. Also, the fluorination catalyst may be expected to be further applied to the production of fluorinated compounds used for polymer materials, medicines and agricultural products.

### DESCRIPTION OF THE PREFERRED EMBODIMENT

The chromium salt as the raw material is one or more chromium salts selected from a halogenated chromium oxide, chromium nitrate, chromium sulfate, halogenated chromium, chromium perchlorate, chromium acetate and chromium acetylacetonate. The chromium salt used in the present invention is preferably chromium fluoride or a halogenated chromium oxide represented by CrOₐX_{b} (wherein X represents a halogen atom, a denotes 0 or 1 to 3 and b denotes 0 or 1 to 6, provided that 3≦a+b≦6) or a mixture of these compounds, and more preferably fluorinated chromium oxide.

The metal salt as the raw material may further include, besides a chromium salt (or chromium oxide) as a major component, one or more other metal salts selected from magnesium salts, aluminum salts, zinc salts, sodium salts, nickel salts, iron salts, cobalt salts, vanadium salts, manganese salts and copper salts or metal oxides which are catalytically active.

These catalytically active other metal salts are preferably halides, nitrates, sulfates, silicates, perchlorate, acetates or organic acid salts though no particular limitation is imposed on the structures of these salts.

The proportion of the chromium salt is 30% by weight to 100% by weight and particularly preferably 50% by weight to 90% by weight based on all metal salts.

There is no particular limitation to the content of the metal salts except for chromium salts. However, in the case of a zinc salt, its content is preferably 0.1% by weight to 10% by weight and more preferably 0.5% by weight to 3% by weight based on zinc. In the case of an aluminum salt, its content is preferably 0.1% by weight to 20% by weight and more preferably 1% by weight to 5% by weight based on aluminum. In the case of a magnesium salt, its content is preferably 0.1% by weight to 10% by weight and more preferably 0.5% by weight to 3% by weight based on magnesium.

The surface area of the metal salt is preferably 90 m²/g or more and more preferably 150 m²/g or more, though no particular limitation is imposed on it.

Although no particular limitation is imposed on a method for the preparation of the metal salt, examples of that method may include a method in which a gel of a metal hydroxide is baked to prepare a metal oxide and a method in which the metal oxide or the like is further treated using hydrogen fluoride to prepare a metal fluoride or fluorinated metal oxide. When, for example, a gel of a metal hydroxide is baked, it is usually baked at 100° C. to 500° C. and preferably 300° C. to 500° C. under a nitrogen atmosphere. In the hydrogen fluoride treatment, it is only required to bring the metal oxide or the like into contact with hydrogen fluoride or with hydrogen fluoride diluted with diluent gas such as nitrogen gas or argon gas. The hydrogen fluoride treatment is carried out at ambient temperature to 500° C. and preferably 50° C. to 350° C. There is no particular limitation to the amount of hydrogen fluoride. Hydrogen fluoride left unremoved can be removed under a nitrogen stream.

If a metal salt containing chromium salt or chromium oxide as its major component is treated by chlorine and/or oxygen, it is changed in chemical composition to thereby obtain a metal salt material suitable as a catalyst. Unexpectedly, the material obtained by chlorine or oxygen treatment was significantly increased in catalytic activity as compared with the untreated metal salt. This is considered to be because the valence number of contained chromium is made higher by the surface treatment since chlorine and oxygen are oxidants. However, there was no sign of an improvement in the catalytic activity of a material treated with fluorine gas that was likewise an oxidant. The reason why high catalytic activity is brought about in this manner by chlorine treatment and/or oxygen treatment has not been clarified. It is however inferred that the chlorine or oxygen treatment changes the surface composition of the metal salt, bringing about high catalytic activity. Here, the change in chemical composition can be confirmed, for example, by EDX analysis (energy distributed type X-ray analysis: the characteristic X-ray of a sample is detected when the surface of the sample placed under vacuum is irradiated with electron rays to make the qualitative and quantitative analysis and analysis of structural elements of the sample).

Examples of the treating agent may include chlorine gas and oxygen gas. Among these gases, chlorine gas is preferable. The temperature in the oxidizing treatment is 100° C. to 500° C. and preferably 300° C. to 400° C. The amount of the treating agent is 1% by weight to 15% by weight and preferably 5% by weight to 10% by weight based on the catalyst.

The material of the present invention is preferably used as a fluorination catalyst for producing fluorides by reacting halides with hydrogen fluoride. As to the reaction, the material of the present invention may be used in liquid-phase or vapor-phase fluorination reactions.

Although no particular limitation is imposed on the halide used as the raw material, the halide is preferably a halogenated alkene or alkane having 1 to 4 carbon atoms and at least one chlorine and more preferably a halide propane represented by the formula CF_{y}Cl_{3-y}CH₂CHF_{w}Cl_{2-w} (wherein w denotes 0 or 1 and y denotes 0 to 3) and having 3 carbon atoms. Specific examples of these halides may include 1,1,1,3,3-pentachloropropane, 1-chloro-3,3,3-trifluoropropene and 1,3,3,3-tetrafluoropropene.

1,1,1,3,3-pentachloropropane can be efficiently converted into 1-chloro-3,3,3-trifluoropropene and 1,3,3,3-tetrafluoropropene (both are useful as the raw material used to produce 1,1,1,3,3-pentafluoropropane) by using the catalyst of the present invention. In this case, the reaction temperature is 100° C. to 450° C. and preferably 200° C. to 300° C. The molar ratio of hydrogen fluoride to 1,1,3,3,3-pentachloropropane is 1:1 to 30:1 and preferably 5:1 to 15:1.

1-chloro-3,3,3-trifluoropropene and 1,3,3,3-tetrafluoropropene can be efficiently converted into HFC-245fa by reaction with hydrogen fluoride in a vapor phase utilizing the catalyst of the present invention. In this case, the reaction temperature is 100° C. to 450° C. and preferably 150° C. to 300° C. The molar ratio of hydrogen fluoride to the substrate is 1:1 to 30:1 and preferably 5:1 to 15:1.

The present invention will be explained in detail by way of examples. However, the following examples are not intended to be limiting of the present invention.

### Example 1

A hydroxide obtained from 360 g of a 10% CrCl₃ solution containing 1 g of ZnCl₂ and 13 g of Al(NO₃)₃.9H₂O by a coprecipitation method was baked and was further treated with HF to obtain a mixed metal fluoride. 15 ml of the mixed metal fluoride was then placed in a reactor made of inconel to allow chlorine to pass through the reactor at 400° C. for 4 hours, followed by removing remaining chlorine by flowing nitrogen.

Next, trans-1-chloro-3,3,3-trifluoropropene (TTCP) and anhydrous hydrogen fluoride (AHF) were supplied to the reactor containing 15 ml of the chlorine treated metal salt through a vaporizer kept at 150° C. The product was washed in a 60° C. warm water stream to remove HF and HCl and allowed to pass through a CaCl₂ drying tube and then measured by GC (GC-14A manufactured by Shimadzu Corporation was used). The results are shown in Table 1.

### Comparative Example 1

The mixed metal fluoride obtained before the chlorine treatment in Example 1 was used to run a fluorination reaction of TTCP in the same manner as in Example 1. The results are shown in Table 1. Both the conversion rate of TTCP and the selectivity of 1,1,1,3,3-pentafluoropropane (HFC-245fa) were lower than those obtained in Example 1.

TABLE 1

| | Chlorine | Reaction | Conversion | TTFP | HFC-245fa | CTCP |
|---|---|---|---|---|---|---|
| | treatment | temp. ° C. | rate of TTCP % | GC area % | | |
| Example 1 | Treated | 220 | 88.1 | 5.6 | 91.8 | 0.8 |
| | | 240 | 87.6 | 7.2 | 89.8 | 0.9 |
| Comparative | Untreated | 220 | 71.6 | 7.9 | 87.2 | 2.3 |
| Example 1 | | 240 | 84.2 | 9.0 | 86.7 | 1.4 |

HF/Raw material = 10/1, CT 5.45 S
TTCP: Trans-1-chloro-3,3,3-trifluoropropene
TTFP: Trans-1,3,3,3-tetrafluoropropene
CTCP: Cis-1-chloro-3,3,3-trifluoropropene
HFC-245fa: 1,1,1,3,3-pentafluoropropane

### Example 2

The operation involving the preparation of the mixed metal fluoride, the chlorine treatment and the fluorination reaction of TTCP was carried out in the same manner as in Example 1 except that 240 g of a 10% CrCl₃ solution containing 0.65 g of ZnCl₂, 1.0 g of MgCl₂ and 8.5 g of Al(NO₃)₃.9H₂O was used as the raw material of the mixed metal fluoride.

The results are shown in Table 2.

### Comparative Example 2

The mixed metal fluoride obtained before the chlorine treatment in Example 2 was used to run a fluorination reaction of TTCP in the same manner as in Example 1. The results are shown in Table 2. Both the conversion rate of TTCP and the selectivity of 1,1,1,3,3-pentafluoropropane (HFC-245fa) were lower than those obtained in Example 2.

TABLE 2

| | Chlorine | Reaction | Conversion | TTFP | HFC-245fa | CTCP |
|---|---|---|---|---|---|---|
| | treatment | temp. ° C. | rate of TTCP % | GC area % | | |
| Example 2 | Treated | 200 | 72.8 | 4.4 | 90.5 | 1.9 |
| | | 220 | 87.8 | 3.7 | 92.6 | 1.3 |
| Comparative | Untreated | 220 | 63.5 | 9.0 | 84.9 | 3.1 |
| Example 2 | | 240 | 82.4 | 8.3 | 86.2 | 2.0 |

HF/Raw material = 10/1, CT 5.45 S

### Example 3

The operation involving the preparation of the mixed metal fluoride, the chlorine treatment and the fluorination reaction of TTCP was carried out in the same manner as in Example 1 except that 240 g of a 10% CrCl₃ solution containing 0.65 g of ZnCl₂ and 8.5 g of Al(NO₃)₃.9H₂O was used as the raw material of the mixed metal fluoride. The results are shown in Table 3.

### Comparative Example 3

The mixed metal fluoride obtained before the chlorine treatment in Example 3 was used to run a fluorination reaction of TTCP in the same manner as in Example 1. The results are shown in Table 3. Both the conversion rate of TTCP and the selectivity of HFC-245fa were lower than those obtained in Example 3.

TABLE 3

| | Chlorine | Reaction | Conversion | TTFP | HFC-245fa | CTCP |
|---|---|---|---|---|---|---|
| | treatment | temp. ° C. | rate of TTCP % | GC area % | | |
| Example 3 | Treated | 200 | 69.9 | 3.9 | 91.3 | 2.7 |
| | | 220 | 86.2 | 3.9 | 92.4 | 1.4 |
| Comparative | Untreated | 220 | 50.8 | 16.6 | 75.1 | 5.1 |
| Example 3 | | 240 | 72.3 | 11.8 | 83.3 | 3.5 |

HF/Raw material = 10/1, CT 5.45 S

### Example 4

The operation involving the preparation of the mixed metal fluoride, the chlorine treatment and the fluorination reaction of TTCP was carried out in the same manner as in Example 1 except that 240 g of a 10% CrCl₃ solution containing 0.65 g of ZnCl₂, 8.5 g of Al(NO₃)₃.9H₂O and 9.0 g of Na₂SiO₃ was used as the raw material of the mixed metal fluoride. The results are shown in Table 4.

### Comparative Example 4

The mixed metal fluoride obtained before the chlorine treatment in Example 4 was used to run a fluorination reaction of TTCP in the same manner as in Example 1. The results are shown in Table 4. Both the conversion rate of TTCP and the selectivity of HFC-245fa were lower than those obtained in Example 4.

TABLE 4

| | Chlorine | Reaction | Conversion | TTFP | HFC-245fa | CTCP |
|---|---|---|---|---|---|---|
| | treatment | temp. ° C. | rate of TTCP % | GC area % | | |
| Example 4 | Treated | 200 | 55.7 | 6.2 | 87.6 | 3.4 |
| | | 220 | 74.9 | 4.0 | 92.1 | 2.7 |
| Comparative | Untreated | 220 | 33.3 | 17.9 | 64.4 | 10.7 |
| Example 4 | | 240 | 65.3 | 10.9 | 78.0 | 5.0 |

HF/Raw material = 10/1, CT 5.45 S

### Example 5

A hydroxide obtained from 360 g of a 10% CrCl₃ solution containing 1 g of ZnCl₂ and 13 g of Al(NO₃)₃.9H₂O by a coprecipitation method was baked and was further treated with HF to obtain a mixed metal fluoride. 15 ml of the mixed metal fluoride was then placed in a reactor made of inconel to allow oxygen to pass through the reactor at 400° C. for 4 hours, followed by removing remaining oxygen by flowing nitrogen.

Next, trans-1-chloro-3,3,3-trifluoropropene (TTCP) and anhydrous hydrogen fluoride (AHF) were supplied to the reactor containing 15 ml of the oxygen treated metal salt through a vaporizer kept at 150° C. The product was washed in a 60° C. warm water stream to remove HF and HCl and allowed to pass through a CaCl₂ drying tube and then measured by GC (GC-14A manufactured by Shimadzu Corporation was used). The results are shown in Table 5.

### Comparative Example 5

The mixed metal fluoride obtained before the chlorine treatment in Example 5 was used to run a fluorination reaction of TTCP in the same manner as in Example 1. The results are shown in Table 5. Both the conversion rate of TTCP and the selectivity of 1,1,1,3,3-pentafluoropropane (HFC-245fa) were lower than those obtained in Example 5.

TABLE 5

| | Oxygen | Reaction | Conversion | TTFP | HFC-245fa | CTCP |
|---|---|---|---|---|---|---|
| | treatment | temp. ° C. | rate of TTCP % | GC area % | | |
| Example 5 | Treated | 200 | 73.0 | 7.7 | 87.8 | 2.0 |
| | | 220 | 88.5 | 8.4 | 89.4 | 0.9 |
| Comparative | Untreated | 220 | 71.6 | 7.9 | 87.2 | 2.3 |
| Example 5 | | | | | | |

HF/Raw material = 10/1, CT 5.45 S

### Comparative Example 6

A hydroxide obtained from 240 g of a 10% CrCl₃ solution containing 0.65 g of ZnCl₂ and 8.5 g of Al(NO₃)₃.9H₂O by a coprecipitation method was baked and was further treated with HF to obtain a mixed metal fluoride. 15 ml of the mixed metal fluoride was then placed in a reactor to carry out fluorine gas treatment at ambient temperature.

Next, trans-1-chloro-3,3,3-trifluoropropene (TTCP) and anhydrous hydrogen fluoride (AHF) were supplied to the reactor containing 15 ml of the fluorine treated metal salt through a vaporizer kept at 150° C. The product was washed in a 60° C. warm water stream to remove HF and HCl and allowed to pass through a CaCl₂ drying tube and then measured by GC (GC-14A manufactured by Shimadzu Corporation was used). The results are shown in Table 6.

### Comparative Example 7

The mixed metal fluoride obtained before the chlorine treatment in Comparative Example 6 was used to run a fluorination reaction of TTCP in the same manner as in Comparative Example 6. The results are shown in Table 6.

Compared with Example 5 and 6, no improvement was seen in the conversion rate of TTCP and the selectivity of 1,1,1,3,3-pentafluoropropane (HFC-245fa) even if fluorinating treatment was carried out.

TABLE 6

| | Fluorine | Reaction | Conversion | TTFP | HFC-245fa | CTCP |
|---|---|---|---|---|---|---|
| | treatment | temp. ° C. | rate of TTCP % | GC area % | | |
| Comparative | Treated | 200 | 47.1 | 21.9 | 70.2 | 8.0 |
| Example 6 | | 220 | 46.8 | 18.8 | 73.2 | 8.0 |
| Comparative | Untreated | 200 | 45.0 | 17.7 | 69.7 | 12.5 |
| Example 7 | | 220 | 67.1 | 16.1 | 79.8 | 4.2 |

### Example 6

EDX analysis (using S4800 manufactured by Hitachi, Ltd. and EMAX Energy EX-320S manufactured by Horiba, Ltd.) of the metal fluoride used as the catalyst in Example 3 was made before and after the chlorine treatment, to find that the chemical composition of the surface of the metal fluoride was different before and after the chlorine treatment as shown in Table 7.

TABLE 7

| Chlorine | Weight ratio to Cr | | |
|---|---|---|---|
| treatment | C/Cr | F/Cr | Cl/Cr |
| Untreated | 0.96 | 0.63 | - (Cr was undetected) |
| Treated | 0.86 | 0.81 | 0.03 |

### Example 7

EDX analysis of the metal fluoride used as the catalyst in Example 5 was made before and after the oxygen treatment, to find that the chemical composition of the surface of the metal fluoride was different before and after the oxygen treatment as shown in Table 8.

TABLE 8

| Oxygen | Weight ratio to Cr | | |
|---|---|---|---|
| treatment | C/Cr | F/Cr | Cl/Cr |
| Untreated | 0.60 | 0.25 | - (Cr was undetected) |
| Treated | 0.78 | 0.21 | - (Cr was undetected) |

### Comparative Example 8

EDX analysis of the metal fluoride used as the catalyst in Comparative Example 6 was made before and after the fluorine treatment, to find that the chemical composition of the surface of the metal fluoride was different before and after the fluorine treatment as shown in Table 9.

TABLE 9

| Fluorine | Weight ratio to Cr | | |
|---|---|---|---|
| treatment | C/Cr | F/Cr | Cl/Cr |
| Untreated | 0.96 | 0.63 | - (Cr was undetected) |
| Treated | 0.91 | 0.90 | 0.02 |

### INDUSTRIAL APPLICABILITY

The fluorination catalyst of the present invention is stable at high temperatures and enables the production of fluorinated compounds such as HFC-245fa and intermediate raw materials used for synthesizing HFC-245fa at a high conversion rate and high selectivity.

Therefore, if the fluorination catalyst of the present invention is used, cooling mediums, detergents and foaming agents that are made of materials substituted for Freon can be produced efficiently. Also, the fluorination catalyst may be expected to be further applied to the production of HFC (hydrofluorocarbon) used for polymer materials, medicines and agricultural products.

What is claimed is:

1. A method for the preparation of a fluorinated alkane or a fluorinated alkene, the method comprising reacting an alkane or an alkene having at least one halogen atom from the group consisting of chlorine, bromine and iodine and 1 to 4 carbon atoms with hydrogen fluoride in the presence of a fluorination catalyst obtained by treating a metal salt containing a chromium salt or chromium oxide with chlorine gas and/or oxygen gas.

2. A method for the preparation of a fluorinated alkane or a fluorinated alkene according to claim 1, wherein the chromium salt is a halogenated chromium oxide, chromium nitrate, chromium sulfate, halogenated chromium, chromium perchlorate, chromium acetate, chromium acetylacetonate or a mixture of these compounds.

3. A method for the preparation of a fluorinated alkane or a fluorinated alkene according to claim 1, wherein the chromium salt is CrCl₃, a halogenated chromium oxide represented by the formula CrOₐX_{b} (wherein X represents a halogen atom, a denotes 0 or 1 to 3 and b denotes 0 or 1 to 6, provided that 3≦a+b≦6) or a mixture of these compounds.

4. A method for the preparation of a fluorinated alkane or a fluorinated alkene according to claim 1, wherein the metal salt is one or more catalytically active metal salts from the group consisting of magnesium salt, an aluminum salt, a zinc salt, a sodium salt, a nickel salt, an iron salt, a cobalt salt, vanadium salt, manganese salt and a copper salt.

5. A method for the preparation of a fluorinated alkane or a fluorinated alkene according to claim 4, wherein the catalytically active metal salt is one selected from the group consisting of
a halide of a member selected from the group consisting of magnesium, aluminum, zinc, sodium, nickel, iron, cobalt, vanadium, manganese and copper,
a nitrate of a member selected from the group consisting of magnesium, aluminum, zinc, sodium, nickel, iron, cobalt, vanadium, manganese and copper,
a sulfate of a member selected from the group consisting of magnesium, aluminum, zinc, sodium, nickel, iron, cobalt, vanadium, manganese and copper,
a silicate of a member selected from the group consisting of magnesium, aluminum, zinc, sodium, nickel, iron, cobalt, vanadium, manganese and copper,
a perchlorate of a member selected from the group consisting of magnesium, aluminum, zinc, sodium, nickel, iron, cobalt, vanadium, manganese and copper,
an acetate of a member selected from the group consisting of magnesium, aluminum, zinc, sodium, nickel, iron, cobalt, vanadium, manganese and copper, and
an organic acid salt of a member selected from the group consisting of magnesium, aluminum, zinc, sodium, nickel, iron, cobalt, vanadium, manganese and copper.

6. A method for the preparation of a fluorinated alkane or a fluorinated alkene according to claim 5, wherein the catalytically active metal salt is a magnesium halide and the content of magnesium is 0.1% by weight to 10% by weight based on all metal salts.

7. A method for the preparation of a fluorinated alkane or a fluorinated alkene according to claim 1, wherein the temperature of the treatment using chlorine gas and/or oxygen gas is 100° C. to 500° C.

8. A method for the preparation of a fluorinated alkane or a fluorinated alkene according to claim 1, wherein the metal salt is prepared by a coprecipitation method or an impregnation method.

9. A method for the preparation of a fluorinated alkane or a fluorinated alkene according to claim 1, wherein the alkane or alkene having at least one halogen atom and 1 to 4 carbon atoms is a propane or propene halide having 3 carbon atoms and represented by the formula CF_{y}Cl_{3-y}CH₂CHF_{w}Cl_{2-w} (wherein w denotes 0 or 1 and y denotes 0 to 3).

10. A method for the preparation of a fluorinated alkene according to claim 1, wherein the fluorinated alkene is 1-chloro-3,3,3-trifluoropropene and/or 1,3,3,3-tetrafluoropropene produced by reacting 1,1,1,3,3-pentachloropropane with hydrogen fluoride in the presence of the fluorination catalyst.

11. A method for the preparation of a fluorinated alkane according to claim 1, wherein the fluorinated alkane is 1,1,1,3,3-pentafluoropropane produced by reacting 1-chloro-3,3,3-trifluoropropene or 1,3,3,3-tetrafluoropropene with hydrogen fluoride in the presence of the fluorination catalyst.
